(19) United States
(12) Patent Application Publication
(10) Pub. No.: US 2002/0029011 A1
(43) Pub. Date: Mar. 7, 2002

(54) Methods and devices for surgery

(76) Inventor: Wallace K. Dyer, Atlanta, GA (US)

Correspondence Address: Mary Anthony Merchant, Ph.D., KILPATRICK STOCKTON LLP, Suite 2800, 1100 Peachtree Street, Atlanta, GA, 30309-4530, US

(21) Appl. No.: 09/942,428

(22) Filed: Aug. 29, 2001

Related U.S. Application Data
(60) Provisional application No. 60/230,529, filed on Sep. 5, 2000.

(51) Int. Cl. A61F 13/00

(52) U.S. Cl. 602/41

(57) ABSTRACT

Methods and devices for use in surgical repair are provided. The present invention comprises methods and devices comprised of laminated materials, preferably comprising polytetrafluoroethelene and fluorinated ethelene propylene. Preferred methods comprise placement of the laminated material for support of areas of the body that have lost tension and natural shape.

### CROSS-REFERENCE TO RELATED APPLICATIONS

This application claims priority to U.S. Provisional Patent Application No. 60/230,529 filed Aug. 5, 2000.

### TECHNICAL FIELD

This application relates to methods and devices for surgery. In particular, the devices are directed to laminated materials and can be used in methods for facial, plastic and reconstructive surgery.

### BACKGROUND OF THE INVENTION

Plastic and reconstructive surgeons have long sought to develop methods and devices to aid in the support of physical structures that have lost their natural tension and support. The most often treated areas include the face, the chest region, the buttocks and other regions that lose tension and sag. Current devices are not adequate in providing a natural-looking structure to prevent such loss of tension in these structures.

The aging process causes gradual and predictable changes in the soft tissue layers of the lower face and neck, the anatomical basis of which has been well documented. Loss of elasticity and fragmentation of collagen results in rhytid formation and skin redundancy. Subcutaneous fat thickens and droops or is ptotic and becomes more noticeable. Stretching of the fascia and musculature results in a loss of the supporting ‘sling’ of the submentum, often resulting in submandibular gland ptosis. Further loss of tone and muscular atrophy results in banding of the medial platysmal borders, blunting of the cervicomental angle and loss of lateral mandibular definition.

The classical rhytidectomy's failure in adequately addressing the consequences of aging in the neck has prompted the development of a number of modifications and adjunctive procedures. These include skin excisions, various lipoplasty techniques, anterior or posteriorly based platysmal transection, resection, or plication procedures, SMAS-platysma flaps, and even suture suspension techniques. However, these modifications have their limitations.

Problems with bowstring contractures and scarring have resulted in the near abandonment of midline skin excision with subsequent Z, W or T-plasty. Liposuction or direct lipocontouring plays an important role in the aging neck. However, fat modification by itself cannot address platysmal banding and often causes an increase in its prominence. Posterior platysmal modification (transection, resection, or plication) is limited by blunting of the lateral mandibular angle and accentuation of anterior irregularities such as skin dimpling. It may also decrease soft tissue support in the submentum. Anteriorly based procedures, whether limited to the upper neck or done in corsett fashion bolsters soft tissue support and improves the cervicomental angle. However, it too fails in improving definition of the lateral mandibular angle. Suspension suture techniques allow for excellent lateral definition but are hampered by suture rupture and skin rippling. Lateral suture suspension also does not allow for subsequent modification.

Perhaps most importantly, current techniques are limited by their inability to combat rebound relaxation inherent to the nature of soft tissues. This inevitably results in the recurrence of banding and submental ptosis requiring in many cases a major revisional procedure. The necessity for revision submentoplasty or neck lift has been noted by many including Kamer who performed a secondary submentoplasty in up to 50% of patients in one study. More recently, Perkins noted the need for revision in 15% of patients undergoing aging neck surgery within 12 months. Rebound tissue relaxation was the main causal factor.

Though many options exist for repair of body structures that have lost their original tone and have begun to sag, none of them provide adequate support. Therefore, what is needed are methods and devices that comprise safe, predictable, and permanent materials for support of body structures. What is particularly needed are methods and devices that provide tensile strength and support for body structures.

### SUMMARY OF THE INVENTION

The present invention generally relates to methods and devices comprising materials that provide support under tension for body structures, and particularly to sling devices that comprise a layered material that provides tensile support. The methods and devices can be used with any body structure that requires added tensile support. Materials that can provide this support are contemplated by the present invention. A preferred embodiment of the present invention comprises a modified cervical sling of reinforced Goretex (SAM), preferably 1.5 cm in width by 30 cm in length. The supporting tensile sling would be comprised of a laminated sandwich, approximately 1 mm to 1.5 mm in thickness with an FEP (fluorinated ethelene propylene) center with an external wrap of e-PTFE SAM graft. This sandwich graft avoids the major disadvantage of slings currently used, which is distensibility (stretch). The added width will provide a more broad-based support for the cervical tissues and still provide the inherent advantages of adjustability and removal, if needed. Such laminated supportive tensile devices can also be used for tensile support of breast tissue, buttocks tissue and lower eyelids, using appropriately sized materials. Additionally, such laminated material can be used in any form. For example, a suture material, made as a Gore-Tex FEP laminate, can be used as a suture alone (tensile support) or, can be woven or braided into structures that provide support for sagging body components or provide support for structures such as the breast and or bladder.

Accordingly, it is an object of the present invention to provide methods and devices that are permanent, resistant to infection, resistant to extrusion and non-antigenic.

Another object of the present invention is to provide methods and devices comprising laminated materials that do not distend or stretch after placement into the body.

It is another object of the present invention to provide methods and devices that provide support for body structures that have lost tension due to aging, injury or other reasons.

Yet another object of the invention is to provide methods and devices that provide tensile strength in contrast to compressive force.

Still another object of the present invention is to provide methods and devices that are mechanically stable with respect to the surrounding tissues.

These and other objects, features and advantages of the present invention will become apparent after a review of the following detailed description and claims.

### BRIEF DESCRIPTION OF THE FIGURES

FIG. 1 is a picture of a preferred embodiment of the device of the present invention.

FIG. 2 shows use of an embodiment of the device used in placement of the device, extending from mastoid to mastoid falling at the apex of the cervicomental angle.

FIG. 3 shows the rear view of placement of an embodiment of the present invention.

### DETAILED DESCRIPTION OF THE INVENTION

The present invention may be understood more readily by reference to the following detailed description included herein. Although the present invention has been described with reference to specific details of certain embodiments thereof, it is not intended that such details should be regarded as limitations upon the scope of the invention. The text of the references mentioned herein, and U.S. Provisional Application No. 60/230,529 filed Aug. 5, 2000 are hereby incorporated in their entireties by reference.

The present invention is directed to methods of tissue repair and novel devices of materials suitable for the repair of body structures that have lost tension. Such materials are permanent, biocompatible, mechanically stable, and replace the tension and shape of the tissues that are repaired. Though the examples provided herein are directed to repair of structures of the cervical area, the present invention contemplates the use of the devices of the present invention for repair of any body structures that require added support.

Although Joulien Bourguet first noted the importance of platysmal manipulation in aesthetic neck surgery in 1928, the difficulty of achieving long lasting results in the neck continues to challenge surgeons more than a half century later. Over the past 3 decades, notable improvements have been made to the classical cervicofacial skinplasty. Surgeons such as Skoog, Jost, Rees, Connel, Baker, Millard, Guerrerosantos, Aston, and more recently, Webster, Feldman, Kamer, and Hamra have brought to the forefront the importance of modifying the deeper tissue layers of the neck. Liposuction and direct lipocontouring have resulted in the accurate removal of unsightly fat accumulation. SMAS (superficial musculoaponeurotic system) and platysma modifications (transection, wedge resection, plication, or rotational flaps) have resulted in the correction of banding, an improved mandibular line and better support for the deeper structures of the neck. These techniques have thus become integral to the modem multi-layered neck lift.

Lipoplasty and platysmal modifications are still inherently limited. Posterior platysmal transection can cause blunting of the lateral mandibular angle and contour irregularities towards the medial platysmal borders. Posterior plication decreases the cervicomental angle, sometimes acutely, but often leads to decreased support of submental soft tissues and submandibular gland prominence. Midline platysmal plication improves the cervicomental angle to a great extent but does not improve lateral mandibular definition. Combining midline and posterior platysmal modification offers perhaps the best surgical outcome. However, this necessitates wide exposure for often short lived results. In the event of low hyoid position, even combination anterior and posterior platysmaplasty fails to obtain an optimal cervicomental angle, 105 to 110 degrees. In addition, aggressive supra-hyoid release procedures, such as transection of the anterior bellies of the digastric, geniohyoid, and myelohyoid to allow movement of the hyoid posteriorly and superiorly secondary to unopposed contraction of the stylohoid muscles, have resulted in only moderate enhancement.

Suture suspension techniques, pioneered by Guerrerosantos, Giampapa, Webster and later modified by Ramirez achieve better definition of the cervicomental angle and lateral mandibular line but are hampered by suture rupture. Skin rippling over the lateral margins of the neck is an additional problem. Also, suture suspension of the platysma does not allow for future tightening. Therefore, revision requires repeat suture suspension.

The chief limitation of current techniques therefore, is their lack of ability to address rebound tissue relaxation. Platysmal modification decreases the amount of recurrent ptosis seen with cervicofacial skinplasty alone. However, forces inherent to the skin that causes rebound relaxation can also be found in muscle and fascia which inevitably results in a recurrence of the submental laxity. These procedures are thus primarily oriented for the short term. They do not allow for simple secondary modification and patients often require revision submentoplasty or secondary rhytidectomy.

Numerous authors including Kamer and Perkins have described in detail the need for revision surgery in the neck with standard procedures. In a recent study by Perkins, rebound relaxation of tissues was the main anatomic factor leading to revision submentoplasty.

The present invention is directed to methods and devices for repairing body structures that have lost tension or need support. Materials that can provide this support are contemplated by the present invention. For example, merseline mesh alone or woven or used in combination with other materials, such as a reinforced Gore-Tex® material, are used. Reinforced Gore-Tex, preferably reinforced with a core of FEP (fluorinated ethelene propylene), can be used in any form. For example, a suture from reinforced Gore-Tex or laminated material can formed into any shape to be used for support of tissues or organs. A preferred embodiment comprises reinforced Gore-Tex with a FEP core (referred to herein as “laminated material”) that has the thickness of 00 suture material. This laminated material is used alone or is woven or braided into a mesh structure that can be used to support tissues or organs. For example, a mesh of laminated material can be used for bladder tacking or for treatment of the ptotic breast.

A preferred embodiment of the present invention comprises use of the devices in methods for repair of neck tissue that has lost tension. This embodiment preferably comprises use of devices comprised of Gore-Tex® and FEP (fluorinated ethelene propylene). Gore-Tex is nontoxic, physically stable, and chemically biocompatible. Gore-Tex is manufactured as an expanded, fibrillated form of polytetrafluoroethelene (PTFE). Sheets of Gore-Tex material have been used in tissue augmentation. Pores between the PTFE fibrils in Gore-Tex® average about twenty-two microns in size and allow limited soft tissue ingrowth. Gore-Tex® evokes a mild chronic inflammatory response and is rapidly surrounded by a thin fibrous capsule. At present, sheets of Gore-Tex have been used in subcutaneous volume augmentation on the chin, malar area, nasal dorsum, nasolabial folds, and lips.

e-PTFE is a suitable material for sling construction because of its soft, natural feel and high biocompatibility. e-PTFE implants make removal possible and provide easy sterilization. Gore-Tex is inert and does not change shape or resorb with time. Additionally, Gore-Tex is not carcinogenic, rarely allergenic, and causes only minimal tissue reaction. Methods of producing Gore-Tex® and products made from Gore-Tex® are disclosed in U.S. Pat. Nos. 4,478,665; 4,482,516; and 4,598,011; all of which are herein incorporated in their entireties.

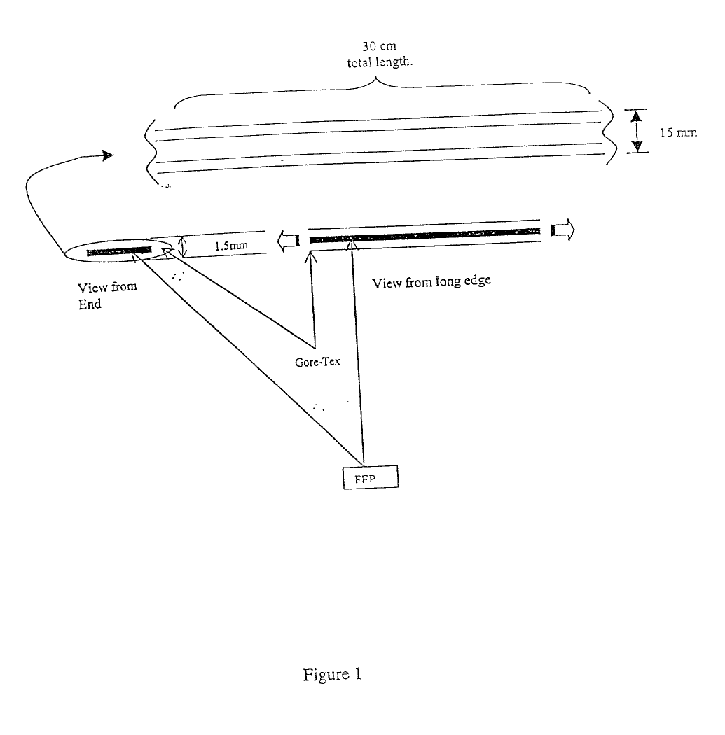

A preferred embodiment of the Gore-Tex® devices of the present invention is a laminated sandwich comprised of a central FEP region surrounded by Gore-Tex®. See FIG. 1 for an illustration of such a preferred embodiment. Such laminated devices are usable in any methods of repair of tissues.

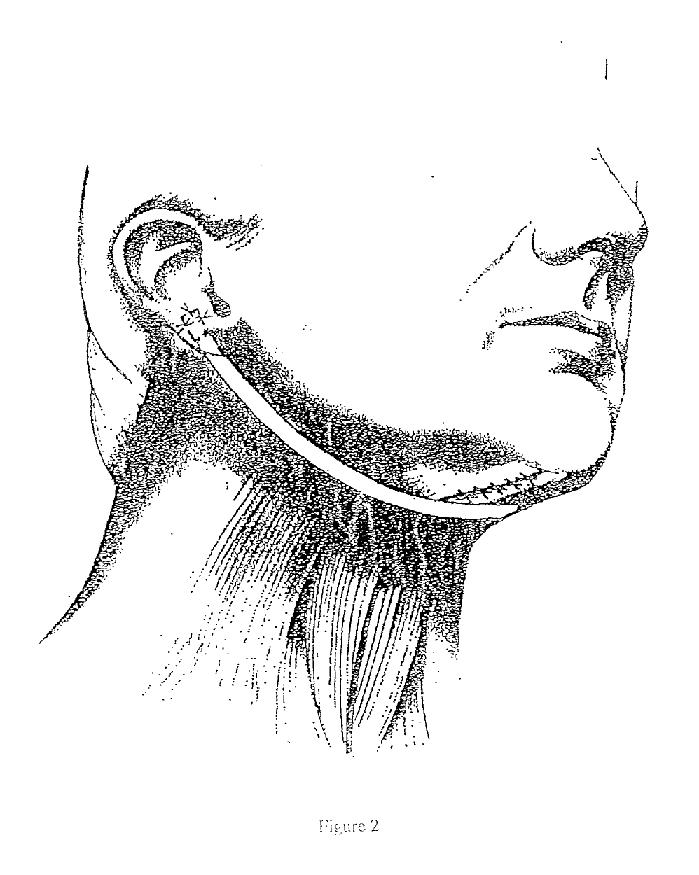

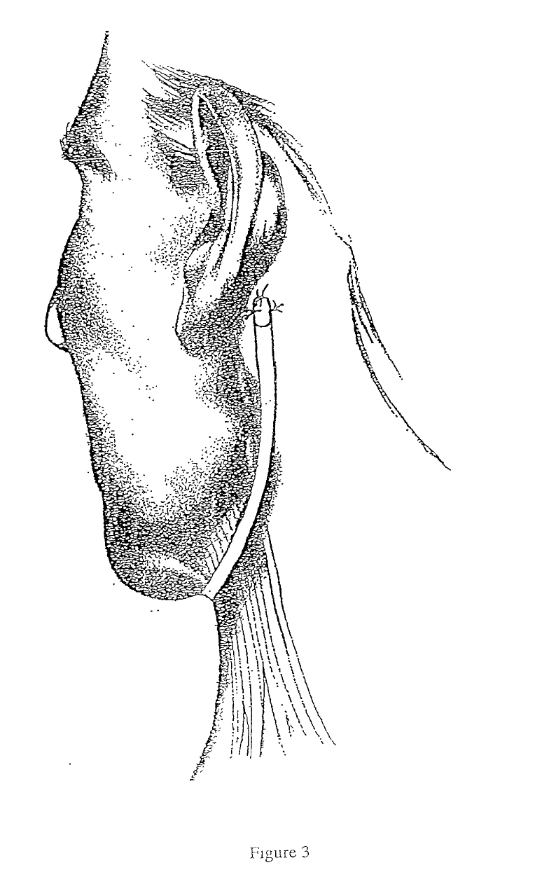

Methods of use of the Gore-Tex® cervical sling device of the present invention are an improvement in lateral suture suspension. The sling is also an improvement in the sense that one preplatysmal strip extends from mastoid to mastoid falling at the apex of the cervicomental angle. The vector in the midline is therefore posterior and superior duplicating the pull of the stylohoid muscles. This is in direct contrast to a Gore-Tex® cervical suspension described by Conrad in 7 patients in which a ‘membrane’ was placed submentally, anterior to the cervicomental angle and anchored to the submental periosteum between the angles of the mandible. Although additional support of the submentum was achieved, recreation of the cervicomental angle was poor and risk to the marginal mandibular nerve increased. The methods of mastoid to mastoid attachment of the device of the present invention results in a more ideal cervicomental angle, avoids lateral blunting, and improves the lateral mandibular line. In those patients with low hyoids, it gives the illusion of more normal hyoid position and obviates more complicated procedures such as suprahyoid muscular release. By virtue of its course across the submandibular triangles, ptotic submandibular glands are also supported. See FIGS. 2 and 3 for illustration of placement of the device of the present invention.

In contradistinction to standard suture suspension, the placement of a 1.5 cm wide strip of the devices of the present invention in the form of a sling allows for simple revision or tightening under local anesthesia. Tightening counters the rebound tissue elasticity by distributing the recurrent submental laxity laterally. Cosmesis of longer duration can thus be achieved.

The physiological nature of the soft tissue—device interaction that allows for sling tightening without formation of lateral pleating is particularly interesting. Histologically, Gore-Tex® sheets used as a facial implant, due to its pore size (22 μm) allows for limited tissue ingrowth. Therefore, tissue ingrowth occurs only when the alloplast is stationary in relation to the surrounding soft tissue envelope. By virtue of normal, horizontal, rotational neck movement after primary surgery, the lateral (fixed portion) of the GoreTex® of preferred embodiments allows for sufficient tissue ingrowth. In contrast, the more medial portion (mid neck), which is in constant motion in relation to the overlying soft tissue incites formation of a fibrocollagenous capsule without tissue ingrowth into the sling.

This allows for movement of the Gore-Tex® laminated device in relation to the overlying soft tissue without tethering or pleating. Since the soft tissue layers (dermis, fat and platysma) essentially fuse after primary surgery, lateral excursion of the Gore-Tex® laminated device, lying in the middle of this soft tissue ‘envelope’ results in tightening of all three layers. The ability of the Gore-Tex® laminated device to be easily ‘tightened’ or revised can be used to advantage in other areas. Recently, Konior who noted rebound tissue relaxation in patients undergoing facial reanimation with Gore-Tex® soft tissue patches was able to advance the implant superiorly and achieve better suspension and correction of the recurrent ptosis. Facial reanimation of longer duration can thus be achieved.

The decision to surgically use an alloplast in an elective situation can initially be a difficult jump in personal philosophy. However, its safe record in multiple areas of surgery has made its inclusion easier. For example, a preferred material, Gore-Tex®, is highly biocompatible and elicits little tissue reaction. It is non-carcinogenic and easily removable. There is no donor site morbidity and an unlimited quantity. It is autoclavable, easy to cut and shape, and its edges are easily beveled. GoreTex® material is chosen for compression uses and is used by most surgical disciplines including general surgery, vascular surgery, obstetrics and gynecology, orthopedics, ophthalmology and urology. In the head and neck, Gore-Tex® material has FDA approval for use in facial reconstruction and augmentation including rhinoplasty, mentoplasty, maxilloplasty, malarplasty, forehead defects, auriculoplasty, orbital repair, facial animation, nasolabial folds and glabellar creases. It is contraindicated for cosmetic lip augmentation, temporomandibular joint reconstruction, cardiovascular defects and dermal placement.

The materials and devices of the present invention can be used for methods in plastic, facial, reconstructive and repair surgery. Any physical bodily structure that needs support can be supported using the present invention. The laminated material, preferably, reinforced Gore-Tex with FEP material, can be used in any form including strips, sheets, sutures, woven structures, patches, shaped items or whatever form is necessary for the surgical procedure. The bodily structures that can be supported include, but are not limited to, eyelids, necks, breasts, buttocks, pectoral regions, bladders, intestines, and other internal organs.

This invention is further illustrated by the following example, which is not to be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims.

### EXAMPLE

All patients are pre-operatively evaluated with respect to the presence or absence of fat accumulation, platysmal weakness or banding, submandibular gland ptosis, cervicomental angle, lateral mandibular angle definition and hyoid position. In addition, patients are stratified along the Dedo classification of cervical abnormalities. A graded surgical approach then dictated the procedure chosen for each patient. Lipoplasty is carried out for excess fat accumulation. Midline platysmal plication with or without lipoplasty is undertaken for those patients with platysmal banding or weakness. If rhytidectomy is indicated, it is done in conjunction with midline platysmal plication and posterior SMAS flap imbrication in all cases with lipoplasty when necessary.

Outcome is judged by a comparison of standardized pre-operative and postoperative photodocumentation at 12 months. Taken into consideration is the acuity of the cervicomental angle, presence of blunting of the angle of the mandible, persistence or recurrence of platysmal banding, submental skin laxity and submandibular gland prominence. This included but is not limited to the visual criteria for successful restoration of the youthful neck as described by Ellenbogen. A grade is then assigned according to the following scale for each patient: poor (minimal to no change), fair (moderate improvement), good (significant improvement), superior (marked improvement).

Some patients had slings that are not the laminated Gore-Tex® device of the present invention, and such slings have to be tightened secondarily. The reason for sling tightening, as well as the interval to tightening from time of primary surgery are noted. At 1 month following sling modification, patients are again graded according to the method mentioned above. Patient satisfaction is similarly assessed.

All charts are carefully reviewed for sling related complications. This included infection, extrusion, seroma formation, induration, sling visibility, sling rupture, sling suture rupture, venous congestion in the neck, and persistent dysphagia or odynophagia. Any and all complications are then recorded.

Surgical Technique: Primary Sling Placement

The patient is seated upright and a suitable horizontal submental crease marked in the midline. If platysmal bands are present, they are demarcated. 3 cm post-lobular incisions are also drawn bilaterally. If a rhytidectomy is planned, standard rhytidectomy incisions obviate the need for these incisions. The patient is then placed supine on the operating table and intravenous anesthesia obtained with noninvasive monitoring secured. The submental region is then infiltrated with a total of 20 ccs of 1% lidocaine with 1:100,000 epinephrine dilution in the subcutaneous plane.

After appropriate preparation and drapage, a 3.5 cm incision is made horizontally in the midline submental crease. Subcutaneous tunneling is then undertaken with the aid of a liposuction cannula. In the event of excess fat, liposuction is then commenced. This is followed by wide subcutaneous scissors dissection between the mandibular borders down to the thyroid prominence in the pre-platysmal plane. Great care is taken to ensure a layer of fat on the skin flap to avoid dermal exposure. Platysmal bands are further defined by removal of fat between the medial edges. Midline plication of the platysma is then completed with a series of interrupted, buried, 2-0 Surgidac sutures from the thyroid cartilage to mentum. If rhyitidectomy is planned, it can now be completed. If not, attention is turned to the post-lobular creases where 3 cm long incisions are made bilaterally. A region of 3 cms is then undermined antero-inferiorly staying superficial to the stemoclaidomastoid fascia. Further dissection is then undertaken at the angle of the mandible connecting the post-auricular dissection to the submandibular triangle.

A 1.5 cm wide, and 1 mm to 1.5 mm thick strip of laminated Gore-Tex® device, soaked in gentamycin solution, is then introduced into the left post auricular incision and withdrawn from the sub-mental incision using a long takahashi forceps. The edge of the Gore-Tex® laminated device is folded upon itself and this double layer is then sutured to the underlying premastoid fascia just behind the ear lobule with 3 interrupted, vertical mattress sutures with 2-0 Surgidac. The opposite end of the GoreTex® laminated device is then passed from the submental incision to the contra-lateral post-auricular incision, again with Takahashi forceps. With adequate lateral traction exerted with a Kelly clamp, the Gore-Tex® laminated device can then be secured to the underlying fascia just behind the ear lobule with an interrupted, vertical mattress 2-0 Surgidac suture after making sure that the strip is snug and in correct position in the midline. The strip naturally falls into the apex of the cervicomental angle at the level of the hyoid. After sharply removing the excess Gore-Tex® laminated material and folding of the distal edge upon itself, three more interrupted 2-0 Surgidac sutures are placed further securing the strip. After achieving careful hemostasis, a suction drain is placed into the neck through a separate stab incision superiorly in the post auricular sulcus. Submental and post-auricular incisions are then closed in layers including interrupted 5-0 vicryl sutures subcutaneously, and a running, interlocking, 5-0 fast absorbing gut suture for the skin. All patients are placed on oral cephalexin or ciprofloxacin for a period of 7 days. The drain should be removed after 48 hours unless drainage exceeded 50 ccs/day.

FIGS. 2 and 3 illustrate the methods of use of the devices of the present invention.

Surgical Technique: Secondary Sling Tightening

The awake patient is seated upright while the 2 edges of the sling postauricularly are palpated. This sling was made from Gore-Tex® material and is not the Gore-Tex® laminated device of the present invention. The Gore-Tex® only material sling has a tendency to stretch and sag over time. The more prominent edge is arbitrarily chosen, marked, and the area infiltrated with 2-3 ccs of 1% lidocaine with 1:100,000 epinephrine dilution. The patient is then laid supine and prepped and draped in the usual sterile fashion.

A 3 cm incision is then made in the inferior portion of the post-auricular crease along the previous suture line. Dissection is undertaken subcutaneously until the edge of the sling is visualized. The Surgidac sutures are carefully lysed and the edge of the material of the sling is grasped with a Kelly clamp. Lateral traction is then exerted, lysing any small proximal adhesions surrounding the material and facilitating its excursion through the subcutaneous tissues. This is continued until submental laxity is adequately resolved and desired tightening achieved. A 2-0 Surgidac suture is then placed in vertical mattress fashion securing the sling material to the underlying fascia. After excision of the excess material (usually about 2 cms), the new edge is folded upon itself and further secured with 2 more 2-0 Surgidac sutures.

After careful hemostasis, the incision is closed in two layers including interrupted 5-0 vicryl sutures subcutaneously, and a running interlocking 5-0 fast absorbing gut suture for the skin. No drainage is necessary. All patients are placed on oral cephalexin or ciprofloxacin for a period of 7 days.

The Gore-Tex® device of the present invention can also be adjusted using the above methods.

Those skilled in the art will now see that certain modifications can be made to the invention herein disclosed with respect to the illustrated embodiments, without departing from the spirit of the instant invention. And while the invention has been described above with respect to the preferred embodiments, it will be understood that the invention is adapted to numerous rearrangements, modifications, and alterations, all such arrangements, modifications, and alterations are intended to be within the scope of the appended claims.

The following references are hereby incorporated by reference herein in their entirety.

1. Webster R C, Smith R C, Smith K F. Face lift 11: Etiology of platysma cording and its relationship to treatment. Head Neck Surg. 1983;6:590-595.

2. Dedo D D. Anatomic basis for sculpturing the neck in myloplasty. Arch Otolaryngol. 1981; 107:439-442.

3. Vistnes L M, Souther S G. The platysma muscle: anatomic considerations for aesthetic surgery of the anterior neck. Clin Plast Surg. 1983; 10:441-448.

4. Micheli-Pellegrini V. Surgical anatomy and dynamics in face lifts. Facial Plast Surg. 1992; 8:1-10.

5. Vistnes L M, Souther S G. The anatomical basis for common cosmetic anterior neck deformities. Ann Plast Surg. 1979;2:381-388.

6. Beeson W H. Extended posterior rhytidectomy. Fac Plast Surg Clin No Amer. 1993; 1:197-216.

7. Adamson J E et al. The surgical correction of the ‘turkey gobble’ deformity. Plast Reconstr Surg. 1964;34:598.

8. Ehlert T K, Thomas J R, Becker F F. Submental W-plasty for correction of ‘turkey gobbler’ deformities. Arch Otol HN Surg. 1990; 116:714-770.

9. Cronin T D, Biggs J J. The T-Z plasty for the male ‘turkey gobbler’ neck. Plast ReconstrSurg. 1971;47:536

10. Millard D R JR, Garst W P, Beck R L, Thompson I D. Submental and submandibular lipectomy in conjunction with a face lift, in the male or female. Plast Reconstr Surg. 1972;49:385-391.

11. Brennan H G. Rhytidectomy. Mandibular and submandibular contouring. Fac Plast Surg Clin NAmer. 1993; 1:181-196.

12. Connell B F. Contouring the neck in rhytidectomy by lipectomy and muscle sling. Plast Reconstr Surg. 1976; 61:3 76-383.

13. Kamer F M, Lefkoff L A. Submental surgery: a graduated approach to the aging neck. Arch Otolaryngol Head Neck Surg. 1991; 117:40-46.

14. Marchac D. Julien Bourget: The pioneer in aesthetic surgery of the neck. Clin Plast Surg. 1983; 10:3.

15. Skoog, T. Plastic Surgery. New Methods and Refinements. Philadelphia, W.B. Saunders Co., 1975.

16. Rees T D. Aesthetic Plastic Surgery, Philadelphia, W. B Saunders Co., 1980.

17. Jost G, Lamouche G. SMAS in rhytidectomy, Aesthetic Plast Surg. 1982;6:69.

18. Connell B F, Gaon A. Surgical correction of aesthetic contour problems of the neck. Clin Plast Surg. 1983; 10:491-505.

19. Aston A J. Platysma-SMAS cervicofacial rhytidoplasty. Clin Plast Surg. 1983; 10:507520.

20. De Souza Pinto E B. Importance of cervicomental complex treatment in rhytidoplasty. Aesthetic Plast Surg. 1981 ;5:69-75.

21. McKinney P. The management of platysma bands. Plast Reconstr Surg. 1996;98:9991006.

22. Feldman J J. Corset platysmaplasty. Clin Plast Surg. 1992; 19:3 69-382.

23. McCurdy J A Jr. A rational approach to the neck in cervicofacial rhytidectomy: a guide to planning skin undermining, platysma surgery, and lipectomy. Laryngoscope. 1984;94:1383-1384.

24. Guerrerosantos J. Neck lift: simplified surgical technique, refinements, and clinical classification. Clin Plast Surg. 1983; 10:379-404.

25. Randall P Skiles M S. The “SMAS sling”: an additional fixation in face lift surgery. Ann Plast Surg. 1984; 12:5-9.

26. Mitz V. The superficial musculoaponeurotic system: a clinical evaluation after 15 years of experience. Facial Plast Surg. 1992;8:11-17.

27. Hamra S. The deep-plane rhytidectomy. Plast Reconstr Surg. 1990;86:53.

28. Kamer F M. Sequential rhytidectomy and the two stage concept. Otol Clin No Amer. 1980;13:305-320.

29. Ramirez, O M. Cervicoplasty: nonexcisional anterior approach. Plast Reconstr Surg. 1997;99:1576-1585.

30. Giampapa V C, Di Bernardo B E. Neck recontouring with suture suspension and liposuction: an alternative for the early rhytidectomy candidate. Aesth Plast Surg. 1995; 19:217-223.

31. Webster R C, Smith R C, Smith K F. Face lift V: Suspending suture for platysmal cording. Head Neck Surg. 1984;6:870-879.

32. Webster R C, Smith R C, Smith K F. Face lift, IV: Use of superficial musculo aponeurotic system suspending sutures. Head and Neck Surgery. 1984;6:780-791.

33. Rees T D, Aston S J. A clinical evaluation of the results of submusculo aponeurotic dissection and fixation in facelifts. Plast Reconstr Surg. 1977;59:851-859.

34. Muenker, R. Problems and variations in cervicofacial rhytidectomy. Facial Plast Surg. 1992;8:33-51.

35. Baker D C. Complications of cervicofacial rhytidectomy. Clin Plast Surg. 1983; 10:543-56

36. Kamer F M, Frankel A S. Isolated submentoplasty; a limited approach to the aging neck. Arch Otol H N Surg. 1997; 123:66-70.

37. Perkins S W, Gibson B. Use of submentoplasty to enhance cervical recontouring in face-lift surgery. Arch Otolaryngol Head NeckSurg. 1993; 119:179-183.

38. Ellenbogen R, Karlin J V. Visual criteria for success in restoring the youthful neck. Plast Reconstr Surg. 1980;66:826-837.

39. Dedo D D. A preoperative classification of the neck for cervicofacial rhytidectomy. Laryngoscope. 1980;90:1894-1896.

40. Guyuron B. Problem neck, hyoid bone, and submental myotomy. Plast Reconstr Surg. 1992;90:830-840.

41. Conrad K, Chapnik J S, Reifen E. E-PTFE (Gore-Tex) suspension cervical facial rhytidectomy. Arch Otol H N Surg. 1993; 119:694-698.

42. Sclafani A P, Thomas J R, Artemus J C, Cooper M H. Clinical and histologic response of subcutaneous expanded polytetrafluorothylene (Gore-Tex) and porous high-density polythylene (Medpor) implants to acute and early infection. Arch Otol H N Surg. 1997; 123:328-336.

43. Konior R J. Facial paralysis reconstruction with Gore-Tex soft tissue patch. Arch Otol H N Surg. 1992; 118:1188-1194.

44. Costantino P. Synthetic biomaterials for soft-tissue augmentation and replacement in the head and neck. Otol Clin No Amer. 1994;27:223-262.

45. Levine B, Ben-nan, W E. The current status of expanded polytetrafluoroethylene (Gore-Tex) in facial plastic surgery. ENTJourn. 1995;74:681-684.

46. Petroff M A, Goode R L, Levet Y. Gore-Tex implants: applications in facial paralysis rehabilitation and soft tissue augmentation. Laryngoscope. 1992; 102:1185-1189.

47. Owsley T G, Taylor C O. The use of Gore-Tex for nasal augmentation: A retrospective analysis of 106 patients. Plast Reconstr Surg. 1994;94:241-251.

48. Stockeld D S, Granstrom L, Backman L, Dahlgren S. Inflammatory response to subcutaneously implanted Marlex and Gore-Tex in massively obese patients. Biomaterials. 1992; 13:261-263.

49. Shoenrock L D, Reppucci A D. Correction of subcutaneous facial defects using GoreTex. Fac Plast Surg Clin NAm. 199412L373-388.

50. Cisneros J L, Singla R. Intradermal augmentation with expanded polytetrafluoroethylene (Gore-Tex) for facial lines and wrinkles. JDermatol Surg Oncol. 1993;19:539-542.

It should be understood, of course, that the foregoing relates only to preferred embodiments of the present invention and that numerous modifications or alterations may be made therein without departing from the spirit and the scope of the invention as set forth in the appended claims.

What is claimed is:

1. A device for tissue repair, comprising a laminated material, having at least two layers, comprising at least one layer of polytetrafluoroethelene and at least one layer of fluorinated ethelene propylene.

2. The device of claim 1 wherein the polytetrafluoroethelene is about 1-3 cm in width.

3. The device of claim 1 wherein the polytetrafluoroethelene is about 20-40 cm in length.

4. The device of claim 1 wherein the polytetrafluoroethelene is 1.5 cm in width and 30 cm in length.

5. The device of claim 1, wherein the laminated material is in a string form.

6. The device of claim 5, wherein the string is woven into a fabric.

7. A method for surgical repair, comprising, using a laminated material to provide support for body structures.

8. The method of claim 7, wherein the laminated material comprises at least two layers, comprising at least one layer of polytetrafluoroethelene and at least one layer of fluorinated ethelene propylene.

9. The method of claim 7, wherein the polytetrafluoroethelene is 1.5 cm in width and 30 cm in length.

10. The method of claim 7, wherein the laminated material is in a string form.

11. The method of claim 10, wherein the string is woven into a fabric.

12. The method of claim 7, wherein the body structure is the breast.

13. The method of claim 7, wherein the body structure is the bladder.

14. The method of claim 7 wherein the body structure is the buttock.
